(19) United States
(12) Patent Application Publication
(10) Pub. No.: US 2009/0130188 A9
(43) Pub. Date: May 21, 2009

(54) Acrylic pressure sensitive adhesives

(75) Inventors: Paul B. Foreman, Somerville, NJ (US); Allison Luciano, Lebanon, NJ (US)

Correspondence Address: NATIONAL STARCH AND CHEMICAL COMPANY, P.O. BOX 6500, BRIDGEWATER, NJ, 08807-3300, US

(73) Assignee: National Starch and Chemical Investment Holding Company

(21) Appl. No.: 11/238,277

(22) Filed: Sep. 29, 2005

(51) Int. Cl. A61F 13/02

(52) U.S. Cl. 424/448; 602/1

(57) ABSTRACT

Solution acrylic polymers suitable for dispersing drugs are particularly useful in transdermal drug delivery applications.

### FIELD OF THE INVENTION

The invention relates to a solution of an acrylic pressure sensitive adhesive in a solvent having specifically defined solubility parameters.

### BACKGROUND OF THE INVENTION

Solution acrylic pressure sensitive adhesives (PSAs) are conventionally used in the manufacture of pressure sensitive adhesive tapes, the adhesive tape comprising a backing and a PSA composition. One field where PSA compositions find wide spread use is the medical segment, e.g., various tapes, plasters, bandages and drug delivery devices. Transdermal drug delivery patches for example can be manufactured by preparing a coating formulation by mixing a solution of the adhesive in a solvent with the drug and any excipients to form a homogeneous solution or suspension; applying the formulation to a substrate (a backing or a release liner) using well known knife, roll or extrusion die coating methods; drying the coated substrate to remove the solvent; and laminating the exposed surface to a release liner or backing.

Organic solvents such as ethyl acetate are conventionally used to prepare solution pressures sensitive adhesive compositions. When used in the manufacture of transdermal patches, the active ingredients, e.g., drug, is dissolved in the solution. In order to achieve constant flux from a transdermal patch it is often necessary to load the adhesive matrix with a concentration of drug above its solubility limit, thereby maintaining a constant concentration gradient across the stratum corneum. When the drug is completely soluble in the adhesive solution it is likely to remain dissolved in the dried matrix as a supersaturated solution of drug in polymer. While this has sometimes been done by design in order to create a much larger concentration gradient, the flux decreases with time since there is no replacement reservoir of drug. Attempts to minimize the effects of depletion by having a large amount of drug, compared with the required dose, dissolved in a thick adhesive matrix result in wasteful, low utilization of the drug.

A major problem with supersaturation is that thermodynamics favors crystallization but the kinetics of this process is slow and unpredictable since nucleation is required. Thus patch developers may try to keep the drug dispersed during the formulating and drying steps. However, many drugs are too soluble in the adhesive's solvent system to permit easy dispersion.

There exists a need in the art for adhesive solutions based on acrylic polymers that overcome the above-described limitations. The currently invention addresses such need.

### SUMMARY OF THE INVENTION

The invention provides an acrylic PSA in an organic solution in which the active ingredient (e.g., drugs) are dispersed rather than fully dissolved. When the active remains at least partially dispersed during the drying process, supersaturation is avoided.

One embodiment of this invention is directed to solution acrylic PSAs comprising a solvent or mixture of solvents which may include minor amounts of an aromatic hydrocarbon. The solvent or solvent mixture has a solubility parameter less than 17.8 but not less than 12, or, greater than or equal to 21.1 but not greater than 40 MPa^{1/2}. Preferably, the solubility parameter will be between about 14 and about 17.7 MPa^{1/2} or between about 23 and about 30 MPa^{1/2}.

Another object of the invention is directed to solution acrylic PSAs comprising a solvent or mixture of solvents having a solubility parameter less than 17.1 but not less than 12, or, greater than about 21.1 but not greater than 40 MPa^{1/2}. Preferably, the solubility parameter will be between about 14 and 17 MPa^{1/2} or between about 23 and 30 MPa^{1/2}.

Still another embodiment of the invention is directed to solution acrylic PSAs comprising a solvent or mixture of solvents having a specifically defined solubility parameter and having an active at least partially dispersed therein.

Yet another embodiment of the invention provides articles manufactured using solution acrylic PSAs comprising a solvent or mixture of solvents having a specifically defined solubility parameter and having an active dispersed therein. The adhesive is well suited for the manufacture of articles such as tapes, plasters, bandages and transdermal drug delivery systems to be adhesively adhered to the skin. Acrylic polymers in high solubility parameter solvents are particularly well suited for delivering highly lipophilic drugs. Conversely, acrylic polymers in low solubility parameter solvents are particularly well suited for delivering more hydrophilic drugs.

A further embodiment is directed to a method of manufacturing articles comprising coating a backing substrate with a solution acrylic PSAs comprising a solvent or mixture of solvents having a specifically defined solubility parameter and having an active at least partially dispersed therein.

### BRIEF DESCRIPTION OF THE DRAWING FIGURE

FIG. 1 is photograph comparing the addition of ketoprofen at 15 wt. % on a solids basis to the polymer solutions of Comparative Example 2 (A) and Example 1 (B).

### DETAILED DESCRIPTION OF THE INVENTION

All references cited herein are incorporated in their entireties by reference.

The invention provides solution acrylic pressure sensitive adhesives that are suitable for dispersing actives which are soluble in conventional adhesive solutions. It has been discovered that solution acrylic PSAs prepared in solvents having a solubility parameter outside the conventional range permit the dispersion of a wider range of drugs than was heretofore possible and that these drugs remain dispersed during the drying process, thus avoiding supersaturation. In one embodiment the solution PSAs of the invention will comprise solvents or blends of solvents, which may include minor amounts of an aromatic hydrocarbon, having a solubility parameter less than 17.8 but not less than 12, or, greater than or equal to 21.1 but not greater than 40 MPa^{1/2}. In another embodiment of the invention, the solution acrylic PSAs of the invention will comprise a solvent or blend of solvents having a solubility parameter less than 17.1 but not less than 12, or, greater than about 21.1 but not greater than 40 MPa^{1/2}.

Table 1 lists the solubility parameter (δ) of various solvents. The solvent blend solubility parameters of solvents were computed from published data on enthalpy of vaporization of the component solvents. Values for petroleum distillates such as commercial hexane or heptane were similarly computed from published data on their component isomers as determined by GC analysis.

The solubility parameter, δ, of a component solvent is herein defined by the equation:
δ=(E_{coh}/Vₘ)^{1/2} (1)
where Vₘ is the molar volume and E_{coh} is the cohesive energy density which is computed from the equation:
E_{coh}=ΔHᵥₐₚ−pΔV ≈ΔHᵥₐₚ−RT (2)
where ΔHᵥₐₚ is the enthalpy of vaporization, R is the Universal Gas Constant and T is the absolute temperature. Values are calculated at T=298K (25° C.). Computation of solubility parameters is discussed in, for example, CRC Handbook of Solubility Parameters and Other Cohesion Parameters, A. F. M. Barton, CRC Press, New York (1983), the disclosure of which is incorporated herein in its entirety. It is noted that a number of semi-empirical, group contribution methods have been proposed for the calculation of solubility parameters. For the purposes of this invention, the values are computed from experimentally determined enthalpy data at 25° C. as defined in equations (1) and (2). It can be shown (see Barton ibid) that the solubility parameter of a mixture of n solvents is given by the sum of δᵢ for each of the individual component solvents weighted by their volume fraction, vᵢ.

$$δ = {\sum }_{i = 1}^{n} {δ}_{i} {v}_{i} \qquad ( 3 )$$

Conventional solution acrylic pressure sensitive adhesives for use in transdermal drug delivery are polymerized in ethyl acetate (δ=18.4 MPa^{1/2}) which has the advantage of being a pure solvent of low toxicity and sufficiently low boiling point to be readily removed in a drying oven. By design (to control molecular weight, for example), or because of a historical prior use in non-drug delivery applications, more complex solvent mixtures are commonly employed. Thus a typical conventional acrylic solution PSA may also contain hydrocarbon solvents and, if crosslinked with a metal alkoxide or chelate, stabilizing alcohols and ketones. Commercially available acrylic solution pressure sensitive adhesives for transdermal drug delivery applications employ solvent mixtures with solubility parameter falling within the range 17.1≦δ≦21.1 MPa^{1/2}. Due to increasing concern about the toxicity of aromatic hydrocarbon solvents such as toluene, solvent mixtures that are essentially free of aromatic hydrocarbons are preferred. Commercially available acrylic solution pressure sensitive adhesives that are free of aromatic hydrocarbons employ solvent mixtures with solubility parameter falling within the range 17.8≦δ<21.1 MPa^{1/2}.

Solution pressure sensitive adhesives that employ a solvent or mixture of solvents having a solubility parameter less than 17.1 but not less than 12, or, greater than or equal to 21.1 but not greater than 40 MPa^{1/2} are encompassed by the invention. In one embodiment, the solubility parameter will be between about 14 and about 17 MPa^{1/2} or between about 23 and about 30 MPa^{1/2}.

Solution pressure sensitive adhesives that employ a solvent, or mixture of solvents having a solubility parameter less than 17.8 but not less than 12, or, greater than or equal to 21.1 but not greater than 40 MPa^{1/2} and optionally comprising not more than about 10 wt. % on total solvent of aromatic hydrocarbons are also encompassed by the invention. In one embodiment, the solubility parameter will be between about 14 and about 17.7 MPa^{1/2} or between about 23 and about 30 MPa^{1/2}.

TABLE 1

| | ΔH | | | | |
|---|---|---|---|---|---|
| | (25° C.) | B.Pt. | M. Wt. | ρ | δ |
| Solvent | kJ/mol | ° C. | g/mol | g/cm³ | MPa^{1/2} |
| C5 Aliphatic | | | | | |
| hydrocarbons | | | | | |
| Cyclopentane | 28.52 | 49.3 | 70.13 | 0.7457 | 16.64 |
| n-Pentane | 26.43 | 36.06 | 72.15 | 0.6262 | 14.42 |
| 2-Methylbutane | 24.85 | 27.8 | 72.15 | 0.6201 | 13.87 |
| C6 Aliphatic | | | | | |
| hydrocarbons | | | | | |
| Cyclohexane | 33.01 | 80.73 | 84.16 | 0.7785 | 16.81 |
| Methylcyclopentane | 31.64 | 71.8 | 84.16 | 0.7486 | 16.11 |
| n-Hexane | 31.56 | 68.73 | 86.18 | 0.6548 | 14.86 |
| 2-Methylpentane | 29.89 | 60.26 | 86.18 | 0.6500 | 14.38 |
| 3-Methylpentane | 30.28 | 63.27 | 86.18 | 0.6598 | 14.59 |
| 2,2-Dimethylbutane | 27.68 | 43.73 | 86.18 | 0.6444 | 13.73 |
| 2,3-Dimethylbutane | 29.12 | 57.98 | 86.18 | 0.6616 | 14.30 |
| Hexane, commercial | | 66-69 | | 0.6680 | 14.96 |
| C7 Aliphatic | | | | | |
| hydrocarbons | | | | | |
| Methylcyclohexane | 35.36 | 100.93 | 98.19 | 0.7694 | 16.05 |
| Ethylcyclopentane | 36.40 | 103.5 | 98.19 | 0.7665 | 16.27 |
| cis-1,3- | 34.20 | 90.8 | 98.19 | 0.7402 | 15.46 |
| Dimethylcyclopentane | | | | | |
| n-Heptane | 36.57 | 98.5 | 100.20 | 0.6837 | 15.25 |
| 2-Methylhexane | 34.87 | 90.04 | 100.20 | 0.6787 | 14.81 |
| 3-Methylhexane | 35.06 | 91.84 | 100.20 | 0.6860 | 14.94 |
| 2,2-Dimethylpentane | 32.42 | 79.2 | 100.20 | 0.6739 | 14.19 |
| 2,3-Dimethylpentane | 34.26 | 89.78 | 100.20 | 0.6951 | 14.85 |
| 2,4-Dimethylpentane | 32.88 | 80.49 | 100.20 | 0.6727 | 14.29 |
| 3,3-Dimethylpentane | 33.03 | 86.06 | 100.20 | 0.6936 | 14.54 |
| 3-Ethylpentane | 35.22 | 93.5 | 100.20 | 0.6982 | 15.10 |
| 2,2,3-Trimethylbutane | 32.05 | 80.86 | 100.20 | 0.6901 | 14.27 |
| Heptane, commercial | | 89-92 | | 0.6856 | 14.88 |
| Aromatic Hydrocarbons | | | | | |
| Toluene | 38.01 | 110.63 | 92.14 | 0.8669 | 18.28 |
| o-Xylene | 43.43 | 144.5 | 106.17 | 0.8802 | 18.43 |
| m-Xylene | 42.65 | 139.1 | 106.17 | 0.8642 | 18.08 |
| p-Xylene | 42.40 | 138.4 | 106.17 | 0.8611 | 17.99 |
| Ethylbenzene | 42.24 | 136.19 | 106.17 | 0.8670 | 18.02 |
| Xylene, commercial | | | | 0.8677 | 18.14 |
| Esters | | | | | |
| Ethyl acetate | 35.60 | 77.11 | 88.11 | 0.9003 | 18.40 |
| Ketones | | | | | |
| Acetone | 30.99 | 56.05 | 58.08 | 0.7899 | 19.69 |
| Methylethylketone | 34.79 | 79.59 | 72.11 | 0.8054 | 19.00 |
| Alcohols | | | | | |
| Methanol | 37.43 | 64.6 | 32.04 | 0.7914 | 29.38 |
| Ethanol | 42.32 | 78.4 | 46.07 | 0.7893 | 26.13 |
| i-Propanol | 45.39 | 82.3 | 60.10 | 0.7855 | 23.68 |

Data source: Handbook of Chemistry and Physics, 78th ed., David R. Lide ed., CRC Press, New York (1997-8)

The acrylic polymer used to prepare the pressure sensitive adhesive is not limiting. The acrylic polymer may or may not be pressure sensitive. As used herein, the term “pressure-sensitive adhesive” refers to a viscoelastic material which adheres instantaneously to most substrates with the application of slight pressure and remains permanently tacky. A polymer is a pressure-sensitive adhesive within the meaning of the term as used herein if it has the properties of a pressure-sensitive adhesive per se or functions as a pressure-sensitive adhesive by admixture with tackifiers, plasticizers or other additives.

The acrylic polymer will typically comprise at least one low glass transition temperature (Tg) alkyl acrylate monomer. Low Tg monomers are those having a homopolymer Tg of less than about 0° C. Preferred alkyl acrylates which may be used to practice the invention have up to about 18 carbon atoms in the alkyl group, preferably from about 4 to about 12 carbon atoms in the alkyl group. Alkyl acrylates for use in the invention include methyl acrylate, butyl acrylate, amyl acrylate, hexyl acrylate, 2-ethylhexyl acrylate, octyl acrylate, isooctyl acrylate, decyl acrylate, dodecyl acrylates, isomers thereof, and combinations thereof. Particularly preferred are butyl acrylate, 2-ethylhexyl acrylate and/or isooctyl acrylate, most preferably 2-ethylhexyl acrylate.

The acrylic polymer may also comprise one or more vinyl ester monomers, particularly preferred is vinyl acetate, and/or may comprise one or more functional monomers. Preferred are carboxy and/or hydroxy functional monomers. Useful carboxylic acids preferably contain from about 3 to about 6 carbon atoms and include, among others, acrylic acid, methacrylic acid, itaconic acid, β-carboxyethyl acrylate and the like. Acrylic acid, methacrylic acid and mixtures thereof are particularly preferred. Examples of hydroxy functional monomers include hydroxyethyl acrylate, hydroxypropyl acrylate, hydroxyethyl methacrylate and hydroxypropyl methacrylate. Preferred for use is hydroxyethyl acrylate.

The adhesives may also contain a nitrogen containing compound, in particularly N-substituted acrylamides or methacrylamides. Examples include N-vinyl pyrrolidone, N-vinyl caprolactam, N-tertiary octyl acrylamide (t-octyl acrylamide), dimethyl acrylamide, diacetone acrylamide, N-tertiary butyl acrylamide, N-isopropyl acrylamide, N-vinyl acetamide and/or N-vinyl formamide.

The acrylic polymer may optionally further comprise other well known comonomers including monomers having a high glass transition temperature (i.e., a Tg greater than about 0° C.). Non-limiting examples include methyl acrylate, methyl methacrylate, ethyl acrylate and/or isobutyl methacrylate. Other comonomers can be used to modify the Tg of the acrylic polymer. Such comonomers include styrene, amides such as acrylamide or methacrylamide, and/or nitriles such as acrylonitrile or cyanoethylacrylate.

Minor amounts, sufficient to increase cohesion without gelling the solution, of crosslinkable multifunctional monomers may be used including, for example, glycidyl methacrylate, allyl glycidyl ether, hexanedioldi(meth)acrylate and the like.

The adhesive compositions of the present invention may also optionally include other monomers such as amines, for example 2-(diethylamino)ethyl methacrylate, to impart functionality to the adhesive or for the purposes of compatibility with a particular drug or excipient.

Adhesives of the invention may also comprise blended polymers wherein the acrylic polymer is blended with and further comprises other types of polymers, including silicone polymers such as polydimethylsiloxane and polymethylphenylsiloxane and rubber polymers such as polyiso-butylene and styrene-isoprene-styrene block copolymer.

In addition to the acrylic polymers, or blends thereof, the adhesive compositions of the invention may optionally comprise a compatible tackifier and/or plasticizer and/or skin permeation enhancers. The acrylic polymer and any tackifier, plasticizer and/or permeation enhancer will be selected and used in amounts effective to produce the desired properties required for the intended end use.

The adhesive compositions may if desired be formulated with a crosslinking agent. Use of a crosslinker adds to the cohesive strength. Preferred are chemical crosslinking agents. In particular, crosslinking agents containing aluminum or titanium may be used to practice the invention. Non-limiting examples include aluminum tris(acetylacetonate) and bis(2,4-pentanedionate-0,0′) bis(2-propanolato) titanium. The crosslinker is typically added in an amount of from about 0.3% to about 2% by weight of the acrylic copolymer.

The compositions of the invention may include other additives known to those skilled in the art to satisfy different properties and meet specific application requirements. Such additives include, for example, antioxidants, fillers, pigments, rheology modifiers, which may be incorporated in minor or larger amounts into the adhesive formulation, depending on the purpose.

The adhesives are useful for delivering drugs through the skin (transdermal) or delivering actives to the skin (dermal). The delivery process may be aided by including a permeation enhancer in the adhesive composition. The level of enhancer in the final solid adhesive is determined by the desired flux of active and may be limited by the requirements for resistance to cold flow. Enhancer loadings up to about 30% on adhesive polymer solids, more typically from about 5 to about 15% may be employed.

It will be obvious to one skilled in the art that the acrylic adhesive polymer composition needs to be designed such that good pressure sensitive adhesive properties are achieved in the final formulation taking into account all added components including the active therapeutic agent, and enhancers, plasticizers, crosslinkers, tackifiers and/or other excipients, if present. It will be further obvious that such excipients may change the solubility parameter of the liquid vehicle for the polymer and that this will need to be taken into account when considering the ability to at least partially disperse a drug in the solution. Partial dispersion, as used herein, means that, upon mixing with the adhesive solution, only a portion of the active ingredient goes into solution, the remainder being suspended as solid particles within the solution. Similarly, after drying the term partial dispersion is intended to mean that a portion of the active is dissolved in the adhesive matrix and the remainder is distributed within the adhesive matrix as solid particles.

The adhesive may be advantageously formulated for use in, for example, medical applications such as wound care, transdermal or dermal drug or cosmeceutical delivery applications. The term transdermal refers to the use of the skin as a portal for the administration of drugs by topical application. The topically applied drug passes into and/or through the skin. Thus “transdermal” is used broadly to refer to the topical administration of a drug which acts locally, i.e., at the surface or within the skin, such as, for example, a blemish patch used to treat acne, and to the topical application of a drug which acts systemically by diffusing through the skin and entering the blood stream.

The pressure sensitive adhesives of the invention having an active dispersed therein may advantageously be used in the manufacture of adhesive articles including, but not limited to, medical tapes and transdermal drug delivery systems including transdermal drug-delivery patches, diagnostic patches, dermal patches for delivery of skin actives and patches for providing a skin care function.

In one embodiment, the adhesive article comprises an adhesive coated on at least one major surface of a backing having a first and second major surface. The adhesive may be present on one or both surfaces of the backing. When the adhesive is coated on both surfaces of the backing, the adhesive on each surface can be the same or different. One embodiment is directed to a transdermal drug delivery system comprising an adhesive layer containing an at least partially dispersed therapeutic agent and a backing layer. In another embodiment, the drug delivery system also comprises a release layer. When the patient peels the release liner from the adhesive and applies the patch, the drug partitions into the stratum corneum (outer skin layer) and permeates through the epidermis and dermis.

Backings which can be used in the practice of this invention include, with or without modification, metal foils, metalized polyfoils, composite foils or films containing poytetrafluoroethylene (TEFLON®)-type materials or equivalents thereof, polyether block amide copolymers, polyurethanes, polyvinylidene chloride, nylon, silicone elastomers, rubber-based poylisobutylene styrene, styrene-butadiene and styrene-isoprene copolymers, polyethylene, polyester, and other such materials used in the art of transdermal drug delivery. Particularly preferred are thermoplastic polymers such as polyolefins, for example polyethylene and polypropylene, and polyesters such as polyethyleneterephthalate.

The term “drug” is to be construed herein in its broadest sense to mean any agent which is intended to produce some therapeutic benefit. The agent may or may not be pharmaceutically active, but will be “bioactive” in the sense that it has an effect on the human body. The agent may be used to treat or alter a condition, which may or may not be a pathological, i.e., a disease state. “Drug”, “bioactive agent,” “preparation,” “medicament,” “therapeutic agent,” “physiological agent” and “pharmaceutical agent” are used interchangeably herein and include substances for use in the diagnosis, cure, mitigation, arrest, treatment or prevention of a condition or disease state or to affect the structure or function of the body. Skin-wellness agents that function to e.g., soften and moisturize are included in this term. The term “treatment” is used broadly to encompass prevention, alteration, cure and control of the condition.

The drug is present in a drug delivery device of the invention in a therapeutically effective amount, i.e., an amount effective to bring about a desired therapeutic result in the treatment of a condition to which the preparation of this invention is to be applied. Effective amount of a drug means a nontoxic but sufficient amount of a drug to provide the selected effect over a specific period of time. The amount that constitutes a therapeutically effective amount varies according to the particular drug incorporated in the device, the condition being treated, any drugs being co-administered with the selected drug, desired duration of treatment, the surface area of the skin over which the device is to be placed, and other components of the drug delivery device. Such an amount is readily determinable by the skilled practitioner.

The drug delivery system of the invention, in addition to the drug, may advantageously also contain an effective amount of a penetration enhancer. An effective amount of a penetration enhancer means an amount that provides a selected increase in membrane permeability, rate of administration and amount of drug.

The device of the invention is placed on the skin and allowed to remain for a time sufficient to achieve or maintain the intended therapeutic effect. The time that constitutes a sufficient time can be selected by those skilled in the art with consideration of the flux rate of the device of the invention and of the condition being treated.

Treatment areas where the delivery device of the invention finds use, and examples of pharmaceutical products which can be incorporated in the devices of the invention, include treatment for incontinence (oxybutinin), central nervous system conditions (methylphenidate), hormone therapy and birth control (estradiol, testosterone, progestin, progesterone, levonorgestrel) cardiovascular (nitroglycerin, clonidine) and cardiotonics (e.g., digitalis, digoxin), pain management or anti-inflammatory (fentanyl, lidocaine, diclofenac, flurbiprofen), cosmetic (benzoyl peroxide, salicylic acid, vitamin C, vitamin E, aromatic oils), antinauseants (scopalamine), smoking cessation (nicotine), antiinflammatory conditions, both steroidal (e.g., hydrocortisone, prednisolone, triamcinolone) and nonsteroidal (e.g., naproxen, piroxicam) treatments, antibacterials (e.g., penicillins such as penicillin V, cephalosporins such as cephalexin, erythromycin, tetracycline, gentamycin, sulfathiazole, nitrofurantoin, and quinolones such as norfloxacin, flumequine, and ibafloxacin), antiprotazoals (e.g., metronidazole), antifungals (e.g. nystatin), calcium channel blockers (e.g. nifedipine, diltiazem), bronchodilators (e.g., theophylline, pirbuterol, salmeterol, isoproterenol), enzyme inhibitors such as collagenase inhibitors, protease inhibitors, elastase inhibitors, lipoxygenase inhibitors, and angiotensin converting enzyme inhibitors (e.g., captopril, lisinopril), other antihypertensives (e.g., propranolol), leukotriene antagonists, anti-ulceratives such as H2 antagonists, antivirals and/or immunomodulators (e.g., 1-isobutyl-1H-imidazo[4,5-c]quinolin-4-amine, 1-(2-hydroxy-2-methyl-propyl)-1H-imidazo[4,5-c]quinoline-4-amine, and acyclovir), local anesthetics (e.g., benzocaine, propofol), antitussives (e.g., codeine, dextromethorphan), antihistamines (e.g., diphenhydramine, chlorpheniramine, terfenadine), narcotic analgesics (e.g., morphine, fentanyl), cardioactive products such as atriopeptides, anticonvulsants (e.g., carbamazine), immunosuppressives (e.g., cyclosporine), psychotherapeutics (e.g., diazepam), sedatives (e.g., phenobarbital), anticoagulants (e.g., heparin), analgesics (e.g., acetaminophen), antimigrane agents (e.g., ergotamine, melatonin, sumatriptan), antiarrhythmic agents (e.g., flecainide), antiemetics (e.g., metaclopromide, ondansetron), anticancer agents (e.g., methotrexate), neurologic agents such as anxiolytic drugs, hemostatics, anti-obesity agents, and the like, as well as pharmaceutically acceptable salts, esters, solvates and clathrates thereof.

Veterinary drugs may also be conveniently applied using the transdermal drug delivery device of the invention, as well as agricultural and horticultural agents. It will be appreciated that transdermal drug delivery in veterinary and horticultural applications enables more exact dosing, and less waste than administration in the food/irrigation water.

The transdermal delivery devices of the invention can be made in the form of an article such as a tape, a patch, a sheet, a dressing or any other form known to those skilled in the art. The dosage system may be produced in any desirable unit form. A circular form is convenient as it contains no corners which might be easily detached from the skin. In addition to having various shapes, the dosage units produced may come in various sizes.

Depending on the design of the patch and the condition to be treated, the patch will remain on the skin for up to an hour or more, up to about one week. In a preferred embodiment, the patch is designed to remain on the skin at the application site for about 24 hours, and to be changed daily. In another preferred embodiment, the patch is to remain on the skin and be changed from once to twice a week. Preferably, the patch will be placed on the skin at a site different from the location of the previously used patches.

The invention will be described further in the following examples, which are included for purposes of illustration and are not intended, in any way, to be limiting of the scope of the invention.

### EXAMPLES

### Example 1

An initial charge containing 32.1 g 2-ethylhexylacrylate, 13.7 g methylacrylate, 3.6 g acrylic acid, 82.5 g cyclohexane, and 0.17 g 2,2′-azobisisobutyronitrile (AIBN) polymerization initiator was prepared and charged to a two liter 4-neck round bottomed flask equipped with stainless steel stirrer, thermometer, condenser, water bath, and slow addition funnels. The initial charge was heated to reflux temperature while stirring. At 10 minutes following the beginning of reflux, a monomer mixture containing 182.5 g 2-ethylhexylacrylate, 77.1 g methylacrylate and 21.0 g acrylic acid and 181.50 g cyclohexane was added uniformly over a period of 2 hours. Also beginning at 10 minutes from the start of reflux, a mixture of 168.3 g cyclohexane and 0.79 g AIBN predissolved in 6.6 g ethyl acetate was added uniformly over a period of 3 hours. Reflux was maintained for a further 2 hours. A solution of 2.5 g of a short half-life polymerization initiator in 72.6 g cyclohexane was then added uniformly over a period of one hour and the flask contents maintained at reflux temperature for a further 2 hours in order to scavenge residual monomers. At the end of the hold period, the flask contents were diluted with 231.1 g cyclohexane, cooled to room temperature and the pressure sensitive adhesive solution discharged. The adhesive solution had a solids content of 37.2%, Brookfield viscosity 26,000 mPa·s and exhibited a slightly hazy appearance. The solubility parameter of the solvent system was calculated from the data in Table 1 to be 16.8 MPa^{1/2}.

### Example 2

### Comparative Example

The adhesive of Example 1 was polymerized in a mixture of ethyl acetate and commercial grade hexane in a 78:22% weight ratio and diluted with a mixture of ethyl acetate, isopropanol and toluene to give an overall solvent blend consisting of 65% ethyl acetate, 12% hexane, 19% isopropanol and 2% toluene by weight. The adhesive solution had a solids content of 34.0%, Brookfield viscosity 2,150 mPa·s and was clear and colorless. The solubility parameter of the solvent system was calculated from the data in Table 1 to be 19.0 MPa^{1/2}.

### Example 3

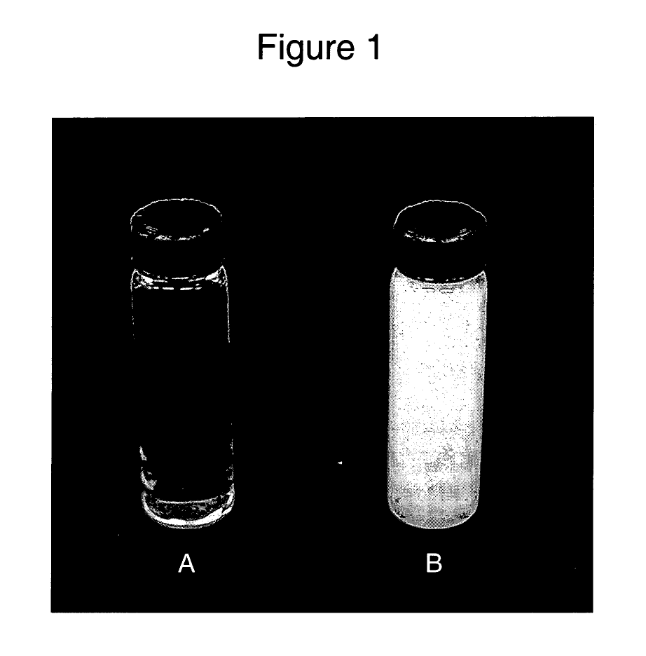

Ketoprofen was added to the adhesive solution of Examples 1 and 2 at 15% on a solids basis. The samples were mixed overnight. As can be seen in FIG. 1, the ketoprofen completely dissolved in the adhesive solution of Example 2 (tube A) whereas in the Example 1 solution (tube B) most of the ketoprofen remained as a dispersed solid.

Adhesive films were prepared by drying for 30 minutes at 60° C. the solutions containing the drug. The film prepared from the cyclohexane adhesive solution (Example 1) contained undissolved crystals of ketoprofen even after drying whereas the film prepared from the conventional adhesive solution (Comparative Example 2) showed no visible crystals indicating that the dissolved drug remains in supersaturated state after drying. This was confirmed by Differential Scanning Calorimetry (DSC) showing zero enthalpy of fusion. In subsequent experiments dried films from Example 1 polymer solution were prepared in which the concentration of ketoprofen was reduced until the enthalpy of fusion of ketoprofen crystals, determined by DSC, approached zero. These measurements placed an upper limit of 2.5 wt. % on the solubility of ketoprofen in the dried adhesive film

### Example 4

The adhesive of Example 1 was polymerized in commercial grade heptane (δ=14.9 MPa^{1/2}) substituting for cyclohexane and yielded a very hazy solution.

### Example 5

An initial charge containing 69.0 g 2-ethylhexylacrylate, 65.1 g n-butylacrylate, 60.0 g t-octylacrylamide 30.0 g methylmethacrylate, 187.4 g cyclohexane, and 0.15 g lauroyl peroxide polymerization initiator was prepared and charged to a two liter 4-neck round bottomed flask equipped with stainless steel stirrer, thermometer, condenser, water bath, and slow addition funnels. The initial charge was heated to reflux temperature while stirring. At 45 minutes following the beginning of reflux, a monomer mixture containing 28.5 g 2-ethylhexylacrylate, 32.4 g n-butylacrylate, 15.0 g methylmethylacrylate was added uniformly over a period of 1 hour. Also beginning at 45 minutes from the start of reflux, a mixture of 41.6 g cyclohexane and 1.08 g lauroyl peroxide was added uniformly over a period of 2 hours. Beginning 190 minutes after initial reflux 107.4 g cyclohexane was uniformly added over 3 hours and the solution was then held at reflux temperature for 6.5 hours. A solution of 1.6 g of a short half-life polymerization initiator in 30.0 g cyclohexane was then added uniformly over a period of one hour and the flask contents maintained at reflux temperature for a further 1 hour in order to scavenge residual monomers. At the end of the hold period, the flask contents were diluted with 16.7 g cyclohexane, cooled to room temperature and the pressure sensitive adhesive solution discharged. The adhesive solution had a solids content of 44.0%, Brookfield viscosity 8,300 mPa·s and exhibited a slightly hazy, colorless appearance. The solubility parameter of the purely aliphatic hydrocarbon solvent was 16.8 MPa^{1/2}.

### Example 6

The polymer of Example 5 was prepared using methylcyclopentane ((δ=16.1 MPa^{1/2}) in place of cyclohexane. A colorless adhesive solution, solids 44.3%, viscosity 890 mPa·s, was obtained having a slightly hazy appearance.

### Example 7

An initial charge containing 52.0 g n-butylacrylate, 24.8 g 2-hydroxyethylacrylate, 3.30 g 2-ethylhexylacrylate, 2.5 g methylmethacrylate, 138.6 g ethanol (solvent), and 0.17 g AIBN was prepared and charged to a 2-L 4-neck round bottom flask equipped with stainless steel stirrer, thermometer, condenser, water bath, and slow addition funnels. The initial charge was heated to reflux while stirring. At 15 minutes from the start of reflux, monomer mix containing 155.9 g n-butyl acrylate, 74.3 g 2-hydroxyethylacrylate, 9.9 g 2-ethylhexylacrylate, 7.4 g methylmethacrylate, and 181.5 g ethanol were simultaneously and uniformly added over a period of 2 hours. Also at 15 minutes from the start of reflux, 51.2 g ethanol and 1.98 g AIBN were simultaneously and uniformly added over a period of 4 hours. At the end of the addition, the flask contents were held at reflux for 1 hour. At 315 minutes from the start of reflux, a solution of 1.2 g of a short half-life initiator in 9.3 g ethanol was added over a period of 1 hour. At the end of the addition, the flask contents were held at reflux for 1 hour. At the end of the hold period, the contents were cooled to room temperature, diluted with 104.0 g ethanol and the solution of pressure sensitive adhesive discharged. A low viscosity, colorless, slightly hazy solution resulted, having a solids content of 40.0%.

### Example 8

### Comparative Example

The polymer of Example 8 was prepared substituting ethyl acetate for ethanol in all steps except the dilution which consisted of 47.3 g ethyl acetate, 37.8 g isopropanol and 18.9 g methanol. The pressure sensitive adhesive solution had solids content of 39.9%, viscosity of 1,200 mPa·s and had a clear, very slightly yellow appearance. The overall solvent mixture was calculated to have solubility parameter δ=19.4 MPa^{1/2}.

### Example 9

Cholesteryl dodecanoate, a model for a highly lipophilic drug, was added at 1% on a solids basis to the solutions of Example 7 and 8. These were then mixed overnight at room temperature. The solution of Example 7 contained a large quantity of undissolved model active whereas in the solution of Example 8 the model active was completely soluble.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A solution of a pressure sensitive adhesive comprising an acrylic polymer and a solvent or mixture of solvents, said solvent or mixture of solvents having a solubility parameter less than 17.1 but not less than 12, or, greater than or equal to 21.1 but not greater than 40 MPa^{1/2}.

2. The solution of claim 1 wherein the solubility parameter is between about 14 and about 17 MPa^{1/2} or between about 23 and 30 MPa^{1/2}.

3. The solution of claim 1 wherein the solvent or solvent mixture comprises cyclohexane.

4. The solution of claim 1 further comprising an active ingredient.

5. The solution of claim 4 wherein the active ingredient is at least partially dispersed in said solution.

6. An article manufactured using the solution of claim 1.

7. The article of claim 6 which is a medical tape.

8. The article of claim 6 which is a drug delivery device.

9. The article of claim 8 wherein the drug is a least partically dispersed within the adhesive matrix.

10. A method of manufacturing an article to be adhesively adhered to the skin comprising coating a backing substrate with the solution acrylic pressure sensitive adhesive of claim 5 and drying the solution.

11. A solution of a pressure sensitive adhesive comprising an acrylic polymer and a solvent or mixture of solvents, said solvent or mixture of solvents having a solubility parameter less than 17.8 but not less than 12, or, greater than or equal to 21.1 but not greater than 40 MPa^{1/2} and optionally comprising an aromatic hydrocarbon, said aromatic hydrocarbon being present at not more than about 10 wt. % on total solvents.

12. The solution of claim 11 wherein the solubility parameter is between about 14 and about 17.7 MPa^{1/2} or between about 23 and 30 MPa^{1/2}.

13. The solution of claim 11 wherein the solvent or solvent mixture comprises cyclohexane.

14. The solution of claim 11 further comprising an active ingredient.

15. The solution of claim 14 wherein the active ingredient is a least partially dispersed in said sloution.

16. An article manufactured using the solution of claim 11.

17. The article of claim 16 which is a medical tape.

18. The article of claim 16 which is a drug delivery device.

19. The article of claim 18 wherein the drug is at least partially dispersed within the adhesive matrix.

20. A method of manufacturing an article to be adhesively adhered to the skin comprising coating a backing substrate with the solution acrylic pressure sensitive adhesive of claim 15 and drying the solution.
